(19) United States
(12) Patent Application Publication
(10) Pub. No.: US 2006/0210499 A1
(43) Pub. Date: Sep. 21, 2006

(54) Mild bleaching agents with increased lightening power

(76) Inventors: Horst Hoeffkes, Duesseldorf (DE); Elisabeth Poppe, Hamburg (DE)

Correspondence Address: DANN DORFMAN HERRELL AND SKILLMAN;A PROFESSIONAL CORPORATION, 1601 MARKET STREET, SUITE 2400, PHILADELPHIA, PA, 19103-2307, US

(21) Appl. No.: 11/362,399

(22) Filed: Feb. 24, 2006

Related U.S. Application Data
(63) Continuation of application No. PCT/EP04/09208, filed on Aug. 17, 2004.

(30) Foreign Application Priority Data
Aug. 26, 2003 (DE) 103 39 163.0

(51) Int. Cl. A61K 8/25

(52) U.S. Cl. 424/62

(57) ABSTRACT

Bleaching agents for keratin-containing fibers, in particular, human hair, comprising (a) at least one peroxo compound and/or at least one alkalinizing agent chosen from ammonium, alkali metal and alkaline earth metal carbonates, hydrogencarbonates and carbamides (b) hydrogen peroxide, and (c) SiO₂ compounds, where the last-mentioned compounds may be optionally hydrated. The agents have an effect of lightening the color of keratin-containing fibers, in particular, human hair. Preferably the bleaching agents are in a pH range of from 4.5 to 9.0. The bleaching agents have a particularly gentle effect on the keratin-containing fibers and the skin.

### CROSS-REFERENCE TO RELATED APPLICATIONS

This application is a continuation under 35 U.S.C. §365(c) and 35 U.S.C. §120 of International Application No. PCT/EP2004/009208, filed Aug. 17,2004. This application also claims priority under U.S.C. §119 of German Patent Application No. DE 103 39 163.0, filed Aug. 26, 2003. Both the International application and the German application are incorporated herein by reference in their entireties.

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### BACKGROUND OF THE INVENTION

(1) Field of the Invention.

The present invention relates on the one hand to bleaching agents for keratin-containing fibers with a pH of from 4.5 to 9.0, comprising peroxo compounds and/or carbonate-, hydrogen carbonate-, or carbamide-containing alkalinizing agents, and hydrogen peroxide and SiO₂ compounds, where the last-mentioned may be optionally hydrated, and on the other hand to the use of optionally hydrated SiO₂ compounds for increasing the lightening power of bleaching agents for keratin-containing fibers, and to a method of lightening keratin-containing fibers using the bleaching agents according to the invention.

Human hair is nowadays treated in diverse ways with hair cosmetic preparations. These include, for example, cleansing the hair with shampoos, care and regeneration with rinses and treatments, and bleaching, coloring and shaping the hair using colorants, tints, waving compositions and styling preparations. In this connection, agents for changing or nuancing the color of head hair play a prominent role.

For permanent, intense colorations with corresponding fastness properties, so-called oxidation colorants are used. Such colorants usually comprise oxidation dye precursors, so-called developer components and coupler components. The developer components form the actual dyes under the influence of oxidizing agents with one another or with coupling with one or more coupler components. The oxidation colorants are characterized by excellent, long-lasting coloring results.

(2) Description of Related Art, Including Information Disclosed Under 37 C.F.R. §§1.97 and 1.98.

In addition, lightening processes, so-called bleaching processes, are often used. The principles of bleaching processes are known to the person skilled in the art and summarized in relevant monographs, e.g., by K. Schrader, Grundlagen und Rezepturen der Kosmetika [Fundamentals and formulations of cosmetics], 2^{nd} Edition, 1989, Dr. Alfred Hüthig Verlag, Heidelberg, or W. Umbach (Ed.), Kosmetik [Cosmetics], 2^{nd} Edition, 1995, Georg Thieme Verlag, Stuttgart, New York.

For lightening or bleaching or human hair—particularly for strand application, solid or paste-like preparations with solid oxidizing agents are usually mixed with a dilute hydrogen peroxide solution directly prior to use. This mixture is then applied to the hair and rinsed out again after a certain contact time.

The above-mentioned application mixture is referred to below as “bleaching agent.” Unless stated otherwise, all of the quantities given refer exclusively to these application mixtures.

Besides hydrogen peroxide, most of the conventional ready-to-use bleaching agents for keratin-containing fibers comprise peroxodisulfate compounds for increasing the lightening power and, upon application to the fibers, have a pH of greater than pH 9. The lightening power is optimal at this basic pH. However, under these conditions, firstly damage of the keratin-containing fibers and secondly, upon application to the subject, skin irritations arise. Lowering the pH is accompanied by less hair damage and skin irritation, but also inevitably by a reduction in the lightening power.

The laid-open specification JP-A-04 279514 relates to hair bleaching agents which have a mild effect on the keratin-containing fibers and the skin at a pH of from 5 to 8. These bleaching agents comprise hydrogen peroxide, persulfate and hydrogen carbonate. However, the lightening power of these bleaching agents does not satisfy the requirements of a high-performance bleaching agent.

### BRIEF SUMMARY OF THE INVENTION

An object of the present invention is therefore to improve the lightening power of bleaching agents for keratin-containing fibers, in particular in a pH range from 4.5 to 9.0, in order to provide more powerful bleaching agents with increased compatibility for the keratin-containing fibers and the skin of the subject.

Surprisingly, it has now been found that the lightening power of bleaching agents for keratin-containing fibers, in particular in a pH range from 4.5 to 9.0, can be increased by adding optionally hydrated SiO₂ compounds.

The present invention therefore firstly provides the use of optionally hydrated SiO₂ compounds for increasing the lightening power of bleaching agents for keratin-containing fibers, in particular in a pH range from pH 4.5 to 9.0.

According to the invention, keratin-containing fibers are understood as meaning furs, wool, feathers and, in particular, human hair.

### BRIEF DESCRIPTION OF THE SEVERAL VIEWS OF THE DRAWING(S)

Not Applicable.

### DETAILED DESCRIPTION OF THE INVENTION

With regard to the optionally hydrated SiO₂ compounds, the present invention is in principle subject to no limitations. Preference is given to salicic acids, oligomers and polymers thereof, and also salts thereof. Preferred salts are the alkali metal salts, in particular the potassium and sodium salts. The sodium salts are very particularly preferred.

The optionally hydrated SiO₂ compounds can be present in different forms. According to the invention, preference is given to using the SiO₂ compounds in the form of silica gels or, particularly and preferably as waterglass. These SiO₂ compounds may sometimes be present in aqueous solution.

According to the invention, very particular preference is given to waterglasses which are formed from a silicate of the formula (SiO₂)ₙ(Na₂O)ₘ(K₂O)ₚ, where n is a positive rational number and m and p, independently of one another, are a positive rational number or are 0, with the provisos that at least one of the parameters m or p is different from 0 and the ratio between n and the sum of m and p is between 1:4 and 4:1.

Besides the components described by the empirical formula, the waterglasses may also comprise further additives, such as, for example, phosphates or magnesium salts, in small amounts.

Waterglasses which are particularly preferred according to the invention are sold, inter alia, by Henkel under the names Ferrosil® 119, Natronwasserglas 40/42, Portil® A, Portil® AW and Portil® W and by Akzo under the name Britesil® C2O.

The present invention secondly provides agents for lightening keratin-containing fibers with a pH of from 4.5 to 9.0, comprising at least one peroxo compound, and hydrogen peroxide and at least one optionally hydrated SiO₂ compound.

Preferably, the agents according to the invention have a pH of from 4.5 to 8.5, very particularly preferably from 5.0 to 8.0.

The bleaching agents according to the invention comprise hydrogen peroxide as a first important component. According to the invention, the hydrogen peroxide is used as a solution or in the form of a solid addition compound of hydrogen peroxide onto inorganic or organic compounds, such as, for example, sodium perborate, sodium percarbonate, magnesium percarbonate, sodium percarbamide, polyvinylpyrrolidone.nH₂O₂ (n is a positive integer greater than 0), urea peroxide and melamine peroxide.

Although even small amounts of the optionally hydrated SiO₂ compounds increase the lightening power, it may be preferred according to the invention to use the optionally hydrated SiO₂ compounds in amounts of from 0.05% by weight to 15% by weight, particularly preferably in amounts of from 0.15% by weight to 10% by weight and very particularly preferably in amounts of from 0.2% by weight to 5% by weight, in each case based on the total agent according to the invention. The quantitative data here in each case give the content of the SiO₂ compounds (without their water content) in the agents.

The bleaching effect of the hydrogen peroxide can additionally be increased by so-called “boosters.” These are usually peroxo compounds which do not represent addition products of hydrogen peroxide onto other components. The choice of peroxo compounds present in the agents according to the invention is subject in principle to no limitations; customary peroxo compounds known to the person skilled in the art are, for example, ammonium peroxydisulfate, potassium peroxydisulfate, sodium peroxydisulfate, ammonium persulfate, potassium persulfate, sodium persulfate, potassium peroxydiphosphate and peroxides, such as magnesium peroxide and barium peroxide. Among these peroxo compounds, which can also be used in combination, the inorganic compounds are preferred according to the invention. Particular preference is given to the peroxydisulfates, in particular ammonium peroxydisulfate.

The peroxo compounds are present in the bleaching agents according to the invention preferably in amounts of 1-40% by weight, in particular in amounts of 2-30% by weight.

The bleaching agents according to the invention are prepared preferably just prior to use on the fibers by combining at least two components. For this purpose, one component A comprising at least one peroxo compound is mixed with a hydrogen peroxide solution as component B, the resulting mixture having a pH of from 4.5 to 9.0. The optionally hydrated SiO₂ compounds are a constituent of at least one of components A and B. However, they are preferably present in component A. It may be preferred to formulate component A as a solid.

Component A in turn can, however, also be prepared from a component A1 and a component A2 by mixing. In this connection, component A1 is preferably in the form of a W/O or O/W emulsion, and component A2 is in the form of a solid comprising at least one peroxo compound.

The concentration of the hydrogen peroxide solution in component B is determined firstly by the legal provisions and secondly by the desired effect; as a rule, 6 to 12% strength solutions in water are used. The quantitative ratios of component A and of component B are here usually in the range 1:1 to 1:2, an excess of component B being chosen particularly if the desired bleaching effect is not to be too marked.

In a further embodiment, the bleaching agents according to the invention comprise an alkalinizing agent. According to the invention the customary alkalinizing agents known to the person skilled in the art for bleaching agents, such as ammonium, alkali metal and alkaline earth metal hydroxides, carbonates, hydrogen carbonates, hydroxycarbonates and carbamides, and also alkali metal phosphates can be used. The alkalinizing agent is preferably present in component A.

In a preferred embodiment, the bleaching agents according to the invention comprise at least one alkalinizing agent chosen from ammonium, alkali metal and alkaline earth metal carbonates, hydrogen carbonates and carbamides. In this embodiment, the pH of the bleaching agent is preferably in a range from pH 7.5 to 9.0, particularly preferably in a range from 7.9 to 8.5.

The invention thirdly relates to agents for lightening keratin-containing fibers, in particular human hair, which comprise at least one alkalinizing agent chosen from ammonium, alkali metal and alkaline earth metal carbonates, hydrogen carbonates and carbamides, at least one optionally hydrated SiO₂ compound and hydrogen peroxide and have a pH in a range from 7.5 to 9.0, in particular from 7.9 to 8.5. The agents according to the invention bring about an acceptable bleaching of the fibers in the presence of these selected alkalinizing agents even without the presence of peroxo compounds. The quality of the bleaching is brought about by the bleach-boosting effect of the optionally hydrated SiO₂ compounds. However, to further increase the bleaching effect, these agents can additionally comprise at least one peroxo compound, preferably in the quantitative range specified above. For the peroxo compounds which can preferably be used, that stated in the second subject-matter of the invention applies. With regard to the optionally hydrated SiO₂ compounds which can preferably be used, at this point reference is explicitly made to the details presented in the first subject-matter of the invention.

The bleaching agents according to the invention are preferably prepared shortly prior to being applied to the fibers by combining at least two components. For this purpose, a component A comprising at least one alkalinizing agent chosen from ammonium, alkali metal and alkaline earth metal carbonates, hydrogen carbonates and carbamides, is mixed with a hydrogen peroxide solution as component B, the resulting mixture having a pH of from 7.5 to 9.0. The optionally hydrated SiO₂ compounds are a constituent of at least one of components A and B. However, they are preferably present in component A. It may be preferred to formulate component A as a solid.

Component A in turn can, however, also be prepared from a component A1 and a component A2 by mixing. Here, component Al is preferably in the form of the W/O or O/W emulsion, and component A2 is in the form of a solid, comprising at least one alkalinizing agent chosen from ammonium, alkali metal and alkaline earth metal carbonates, hydrogen carbonates and carbamides.

The concentration of hydrogen peroxide solution in component B is determined firstly by legal provisions and secondly by the desired effect; as a rule, 6 to 12% strength solutions in water are used. The quantitative ratios of component A and of component B here are usually in the range 1:1 to 1:2, an excess of component B being chosen particularly if the desired bleaching effect is not to be too marked.

All of the data below refer equally to the agents according to the invention of the second and third subject-matter of the invention.

The bleaching agents according to the invention comprise the alkalinizing agents preferably in amounts of from 1 to 25% by weight, in particular 1.5 to 15% by weight.

As additional bleach boosters, the agents according to the invention can preferably comprise at least one compound chosen from acetic acid, lactic acid, tartaric acid, citric acid, salicylic acid and ortho-phthalic acid.

In a further embodiment, the bleaching agents according to the invention additionally comprise structure-improving active ingredients. Such active ingredients which improve the hair structure are vitamins and derivatives or precursors thereof. According to the invention, particular preference is given to panthenol and its physiologically compatible derivatives. Such derivatives are, in particular, the esters and ethers of panthenol, and cationically derivatized panthenols. Individual representatives are, for example, panthenol triacetate, panthenol monoethyl ether and its monoacetate, and also the cationic panthenol derivatives disclosed in WO 92/13829 A1. A panthenol derivative preferred according to the invention is also its precursor pantolactone. Panthenol is preferred within this group. A further example of a structure-improving vitamin is pyridoxine (vitamin B6).

In addition, polyvinylpyrrolidone (PVP) is also known for its fiber structure-improving properties and is preferred according to the invention.

Further structure-improving compounds which are particularly effective according to the invention are the aldehydes. Particularly preferred examples are formaldehyde and formaldehyde-cleaving compounds, such as, for example, methoxymethyl esters, dimethylol(thio)urea derivatives, oxazolidine derivatives, N-hydroxymethylmaleimide, hexamethylenetetramine and its derivatives, hydantoin derivatives, pyridinium-substituted dimethyl ethers, imidozolidenylurea derivatives, isothiazolinones, 2-bromo-2-nitropropanediol and 5-bromo-5-nitro-1,3-dioxane. Further particularly preferred aldehydes are acetaldehyde, glyoxal, glycerin aldehyde and glutardialdehyde.

A further suitable group of structure-improving active ingredients are plant extracts.

Usually, these extracts are prepared by extraction of the total plant. In individual cases, however, it may also be preferred to produce the extracts exclusively from flowers and/or leaves of the plant.

With regard to the plant extracts which can be used according to the invention, reference is made in particular to the extracts which are listed in the table starting on page 44 of the third edition of the introduction to the ingredients declaration of cosmetic compositions, published by the Industrieverband Körperpflege- und Waschmittel e.V. [German Cosmetic, Toiletry, Perfumery and Detergent Association] (IKW), Frankfurt.

According to the invention, the extracts of oak bark, stinging nettle, hamamelis, hops, chamomile, burdock, horsetail, hawthorn, linden blossom, almond, aloe vera, fir needle, roast chestnut, sandalwood, juniper, coconut, mango, apricot, lemon, wheat, kiwi, melon, orange, grapefruit, sage, rosemary, birch, mallow, lady's smock, wild thyme, yarrow, thyme, Melissa, restharrow, coltsfoot, marshmallow, meristem, green tea, ginseng and ginger root are particularly preferred.

Particular preference is given to the extracts from oak bark, stinging nettle, hamamelis, hops, chamomile, burdock, horsetail, linden blossom, almond, aloe vera, coconut, mango, apricot, lemon, wheat, kiwi, melon, orange, grapefruit, sage, rosemary, birch, lady's smock, wild thyme, yarrow, restharrow, meristem, green tea, ginseng and ginger root.

Of very particular suitability for the compositions according to the invention are the extracts of almond, aloe vera, coconut, mango, apricot, lemon, wheat, kiwi, melon and green tea.

Extractants which can be used for producing the specified plant extracts are water, alcohols, and mixtures thereof. Among the alcohols, preference is given here to lower alcohols, such as ethanol and isopropanol, but in particular polyhydric alcohols, such as ethylene glycol and propylene glycol, either as the sole extractant or in a mixture with water. Plant extracts based on water/propylene glycol in the ratio 1:10 to 10:1 have proven to be particularly suitable.

According to the invention, the plant extracts can either be used in pure form or in dilute form. If they are used in dilute form, they usually comprise about 2-80% by weight of active substance and, as solvent, the extractant or extractant mixture used during their extraction.

In addition, it may be preferred to use mixtures of two or more, in particular of two, different plant extracts in the agents according to the invention.

According to the invention, as structure-improving active ingredients, preference is likewise given to honey extracts. These extracts are obtained in an analogous way to the plant extracts and comprise usually 1-10% by weight, in particular 3-5% by weight, of active substance. Water/propylene glycol mixtures may also be extractants which are preferred here.

Further structure-improving active ingredients are protein hydrolysates, in particular elastin, collagen, keratin, milk protein, soya protein, almond protein and wheat protein hydrolysates, their condensation products with fatty acids, and also quaternized protein hydrolysates. Particular preference is given to greatly degraded keratin hydrolysates with molar masses in the range from 400 to 800. In addition, quaternized protein hydrolysates, as are sold, for example, under the trade names Gluadin® WQ (INCI name: Laurdimonium Hydroxypropyl Hydrolyzed Wheat Protein) and Crotein® Q (INCI name: Hydroxypropyltrimonium Hydrolyzed Collagen), are particularly preferred according to the invention.

Besides the quaternized protein hydrolysates, quaternary polymers also represent structure-improving compounds preferred according to the invention. Particular preference is given to the polymers which are sold under the trade names Mirapol® A15 (INCI name: Polyquaternium-2), Onamer® M (INCI name: Polyquaternium-1) and Merquat® 100 (INCI name: Polyquaternium-6).

Further fiber structure-improving active ingredients are mono-, di- and oligosaccharides, such as, for example, glucose, galactose, fructose, fruit sugar, sucrose and lactose. In addition, according to the invention, it is also possible to use derivatives of these pentoses, hexoses and heptoses, such as the corresponding aldonic and uronic acids (sugar acids), sugar alcohols, sugar amines, such as, for example, N-glucosamine, and glycosides, and also pentoses, hexoses and heptoses etherified with C₄-C₃₀-fatty alcohols. According to the invention, the sugar acids can be used in free form, in the form of their salts, preference being given to calcium, magnesium and zinc salts, and in the form of their esters or lactones. Preferred sugar acids are gluconic acid, glucono-γ-lactone, lactobionic acid, glucuronic acid and its mono- or dilactones, pangamic acid, sugar acid, mannosaccharic acid and its mono- or dilactones, and mucic acid and its mono- or dilactones. Preferred sugar alcohols are sorbitol, mannitol and dulcitol. Preferred glycosides are the methylglucosides. Glucose, N-glucosamine and gluconic acid are particularly preferred from this group.

For the purposes of the present invention, certain amino acids can also be used as active ingredients which improve hair structure. Examples are the amino acids serine, threonine and tyrosine described in DE-195 22 569, which is hereby expressly incorporated by reference. In addition, derivatives of serine, such as, for example, serine phosphate, are also preferred according to the invention. A further structure-improving amino acid is lysine. Serine is a particularly preferred active ingredient which improves fiber structure.

Certain acids, in particular a-hydroxycarboxylic acids, and their salts can likewise be used for improving structure. Structure-improving acids preferred according to the invention are lactic acid, malic acid, tartaric acid, glyceric acid and maleic acid. Lactic acid is particularly preferred. In addition, specific phosphonic acids and their salts improve the structure of keratin-containing fibers. Phosphonic acids preferred according to the invention are n-octylphosphonic acid and n-decylphosphonic acid.

Furthermore, lipid-soluble ester alcohols or ester polyols are known for their structure-improving effect. They are regarded as being lipid-soluble if 5% by weight of these products dissolve in cetyl alcohol at 80° C. to give a clear solution.

The ester alcohols or ester polyols suitable according to the invention are obtainable by reacting an epoxy fatty acid ester with water for mono- or polyhydric alcohols having 1-10 carbon atoms with opening of the epoxide ring and formation of a vicinal dihydroxyethyl or hydroxyalkoxyethyl group. The epoxy fatty acid ester here can also be an epoxidation product of a technical-grade fatty acid ester with fractions of saturated fatty acids. The epoxide oxygen content should, however, be at least 3% by weight, preferably 5-10% by weight.

The epoxy fatty acid esters here are either epoxidized fatty acid esters of monohydric alcohols, i.e., for example, epoxidized oleic acid methyl ester, linoleic acid methyl ester, ricinoleic acid methyl ester or epoxidized fatty acid esters of polyhydric alcohols, e.g., glycerol monooleate or propylene glycol monooleate or epoxidized fatty acid triglycerides, e.g., oleic acid triglyceride or unsaturated oils, such as, for example, olive oil, soya oil, sunflower oil, linseed oil, rapeseed oil.

Of particular interest from an industrial point of view are primarily unsaturated fatty acid methyl ester epoxides of unsaturated plant fatty acids. Thus, as ester polyol, the reaction product of a plant oil fatty acid methyl ester epoxidate with a polyol having 2-6 carbon atoms and 2-6 hydroxyl groups is particularly preferred. Polyols which may be present here are, for example, ethylene glycol, 1,2-propylene glycol, 1,3-propylene glycol, butanediol, pentanediol, hexanediol, glycerol, trimethylolpropane, pentaerythritol, sorbitol or diglycerol.

Of particularly good suitability here for the bleaching agents according to the invention as ester polyol is the reaction product of a plant fatty acid methyl ester epoxidate with trimethylpropane and with a hydroxyl number of 350-450. Such a product based on soya oil fatty acid methyl ester epoxide and trimethylolpropane is available under the trade name Sovermol® 760.

In addition, vitamin B3 can be used as structure-improving active ingredient. This name often covers the compounds nicotinic acid and nicotinamide (niacinamide). According to the invention, preference is given to nicotinamide.

For the purposes of the present invention, it is also possible to use vitamin H as structure-improving active ingredient. Vitamin H refers to the compound (3aS, 4S, 6aR)-2-oxohexahydrothienol[3,4-d]imidazole-4-valeric acid, for which, however, the trivial name biotin has in the meantime become accepted.

Structure-improving active ingredients which are particularly preferred according to the invention are chosen from panthenol, physiologically compatible panthenol derivatives, mono-, di- and oligosaccharides, serine, glyceric acid, niacinamide, vitamin B6, polyvinylpyrrolidone, gluconic acid, biotin and the lipid-soluble ester alcohols or ester polyols.

The agents according to the invention comprise the structure-improving active ingredients preferably in amounts of from 0.1 to 5% by weight, particularly preferably in amounts of from 0.2 to 2% by weight.

In a preferred embodiment of the present invention, the agents further comprise a magnesium compound. The agents according to the invention can be further optimized with regard to their structure-retaining properties by adding Mg²⁺ cations. Preferred magnesium compounds are inorganic and organic Mg²⁺ salts, such as, for example, the halides, the carbonates and hydrogen carbonates, the acetate and the citrate.

The agents according to the invention can furthermore comprise all active ingredients, additives and auxiliaries known for such preparations. In many cases, the bleaching agents comprise at least one surfactant, where in principle either anionic or zwitterionic, ampholytic, nonionic and cationic surfactants are suitable. In many cases, however, it has proven to be advantageous to choose the surfactant from anionic, zwitterionic or nonionic surfactants.

Suitable anionic surfactants in preparations according to the invention are all anionic surface-active substances suitable for use on the human body. These are characterized by a water-solubilizing, anionic group, such as, for example, a carboxylate, sulfate, sulfonate or phosphate group, and a lipophilic alkyl group having about 10 to 22 carbon atoms. In addition, glycol or polyglycol ether groups, ester, ether and amide groups, and hydroxyl groups may be present in the molecule. Examples of suitable anionic surfactants are, in each case in the form of the sodium, potassium and ammonium and the mono-, di- and trialkanolammonium salts having 2 or 3 carbon atoms in the alkanol group,
linear fatty acids having 10 to 22 carbon atoms (soaps),
ethercarboxylic acids of the formula R—O—(CH₂—CH₂O)ₓ—CH₂—COOH, in which R is a linear alkyl group having 10 to 22 carbon atoms and x=0 or 1 to 16,
acyl sarcosides having 10 to 18 carbon atoms in the acyl group,
acyl taurides having 10 to 18 carbon atoms in the acyl group,
acyl isothionates having 10 to 18 carbon atoms in the acyl group,
sulfosuccinic mono- and dialkyl esters having 8 to 18 carbon atoms in the alkyl group and sulfosuccinic monoalkyl polyoxyethyl esters having 8 to 18 carbon atoms in the alkyl group and 1 to 6 oxyethyl groups,
linear alkanesulfonates having 12 to 18 carbon atoms,
linear alpha-olefinsulfonates having 12 to 18 carbon atoms,
alpha-sulfo fatty acid methyl esters of fatty acids having 12 to 18 carbon atoms,
alkyl sulfates and alkyl polyglycol ether sulfates of the formula R—O(CH₂—CH₂O)ₓ—SO₃H, in which R is a preferably linear alkyl group having 10 to 18 carbon atoms and x=0 or 1 to 12,
mixtures of surface-active hydroxysulfonates as in DE-A-37 25 030,
sulfated hydroxyalkyl polyethylene and/or hydroxyalkylene propylene glycol ethers as in DE-A-37 23 354,
sulfonates of unsaturated fatty acids having 12 to 24 carbon atoms and 1 to 6 double bonds as in DE-A-39 26 344,
esters of tartaric acid and citric acid with alcohols, which constitute addition products of about 2-15 molecules of ethylene oxide and/or propylene oxide onto fatty alcohols having 8 to 22 carbon atoms.

Preferred anionic surfactants are alkyl sulfates, alkyl polyglycol ether sulfates and ethercarboxylic acids having 10 to 18 carbon atoms in the alkyl group and up to 12 glycol ether groups in the molecule, and in particular salts of saturated and in particular unsaturated C₈-C₂₂-carboxylic acids, such as oleic acid, stearic acid, isostearic acid and palmitic acid.

Zwitterionic surfactants is the term used to refer to those surface-active compounds which carry in the molecule at least one quaternary ammonium group and at least one —COO⁽⁻⁾ or —SO₃⁽⁻⁾ group. Particularly suitable zwitterionic surfactants are the so-called betaines, such as the N-alkyl-N,N-dimethylammonium glycinates, for example cocoalkyldimethylammonium glycinate, N-acylaminopropyl-N,N-dimethylammonium glycinate, for example cocoacylaminopropyldimethylammonium glycinate, and 2-alkyl-3-carboxymethyl-3-hydroxyethylimidazolines having in each case 8 to 18 carbon atoms in the alkyl or acyl group, and cocoacylaminoethyl hydroxyethylcarboxymethyl glycinate. A preferred zwitterionic surfactant is the fatty acid amide derivative known under the INCI name Cocamidopropyl Betaine.

Ampholytic surfactants are understood as meaning those surface-active compounds which, apart from a C₈₋₁₈-alkyl or acyl group in the molecule, contain at least one free amino group and at least one —COOH or —SO₃H group and are capable of forming internal salts. Examples of suitable ampholytic surfactants are N-alkylglycines, N-alkylpropionic acids, N-alkylaminobutyric acids, N-alkylaminodipropionic acids, N-hydroxyethyl-N-alkylpropylglycines, N-alkyltaurines, N-alkylsarcosines, 2-alkylaminopropionic acids and alkylaminoacetic acids having in each case about 8 to 18 carbon atoms in the alkyl group. Particularly preferred ampholytic surfactants are N-cocoalkylaminopropionate, cocoacylaminoethylaminopropionate and C₁₂₋₁₈-acylsarcosine.

Nonionic surfactants comprise, as hydrophilic group, e.g., a polyol group, a polyalkylene glycol ether group or a combination of polyol and polyglycol ether group. Such compounds are, for example,
addition products of from 2 to 30 mol of ethylene oxide and/or 0 to 5 mol of propylene oxide onto linear fatty alcohols having 8 to 22 carbon atoms, onto fatty acids having 12 to 22 carbon atoms and onto alkylphenols having 8 to 15 carbon atoms in the alkyl group,
C₁₂₋₂₂ fatty acid mono- and diesters of addition products of from 1 to 30 mol of ethylene oxide onto glycerol,
C₈₋₂₂-alkyl mono- and oligoglycosides and ethoxylated analogues thereof,
addition products of from 5 to 60 mol of ethylene oxide onto castor oil and hydrogenated castor oil,
addition products of ethylene oxide onto sorbitan fatty acid esters
addition products of ethylene oxide onto fatty acid alkanolamides.

Examples of the cationic surfactants which can be used in the bleaching agents according to the invention are, in particular, quaternary ammonium compounds. Preference is given to ammonium halides, such as alkyltrimethylammonium chlorides, dialkyldimethylammonium chlorides and trialkylmethylammonium chlorides, e.g., cetyltrimethylammonium chloride, stearyltrimethylammonium chloride, distearyldimethylammonium chloride, lauryldimethylammonium chloride, lauryldimethylbenzylammonium chloride and tricetylmethylammonium chloride. Further cationic surfactants which can be used according to the invention constitute the quaternized protein hydrolysates.

Likewise suitable according to the invention are cationic silicone oils, such as, for example, the commercially available products Q2-7224 (manufacturer: Dow Corning; a stabilized trimethylsilylamodimethicone), Dow Corning 929 emulsion (comprising a hydroxylamino-modified silicone, which is also referred to as amodimethicone), SM-2059 (manufacturer: General Electric), SLM-55067 (manufacturer: Wacker), and Abil®-Quat 3270 and 3272 (manufacturer: Th. Goldschmidt; diquaternary polydimethylsiloxanes, quaternium-80).

Alkylamidoamines, in particular fatty acid amidoamines, such as the stearylamidopropyldimethylamine obtainable under the name Tego Amid® S 18, are characterized not only by a good conditioning effect, but specifically by their good biodegradability. Likewise of very good biodegradability are quaternary ester compounds, so-called “ester quats.” such as the methylhydroxyalkyldialkoyloxyalkylammonium methosulfates sold under the trade name Stepantex®, and also the products sold under the trade name Dehyquart®, such as Dehyquart® AU46.

One example of a quaternary sugar derivative which can be used as cationic surfactant is the commercial product Glucquat® 100, according to INCI nomenclature a “Lauryl Methyl Gluceth-10 Hydroxypropyl Dimonium Chloride.”

The compounds with alkyl groups used as surfactants may in each case be uniform substances. It is, however, usually preferred when preparing these substances to start from native vegetable or animal raw materials, giving mixtures of substances with varying alkyl chain lengths which depend on the particular raw material.

Surfactants which constitute addition products of ethylene oxide and/or propylene oxide onto fatty alcohols or derivatives of these addition products which may be used are either products with a “normal” homologue distribution, or those with a narrowed homologue distribution. In this connection, “normal” homologue distribution is understood as meaning mixtures of homologues which are obtained during the reaction of fatty alcohol and alkylene oxide using alkali metals, alkali metal hydroxides or alkali metal alkoxides as catalysts. By contrast, narrowed homologue distributions are obtained if, for example, hydrotalcites, alkaline earth metal salts of ethercarboxylic acids, alkaline earth metal oxides, hydroxides or alkoxides are used as catalysts. The use of products with a narrowed homologue distribution may be preferred.

In addition, the bleaching agents according to the invention can preferably also comprise a conditioning active ingredient chosen from the group which is formed by cationic surfactants, cationic polymers, alkylamidoamines, paraffin oils, vegetable oils and synthetic oils.

As conditioning active ingredients, cationic polymers may be preferred. These are generally polymers which comprise a quaternary nitrogen atom, for example in the form of an ammonium group.

Preferred cationic polymers are, for example,
quaternized cellulose derivatives, as are commercially available under the names Celquat® and Polymer JR®. The compounds Celquat® H 100, Celquat® L200 and Polymer JR® 400 are preferred quaternized cellulose derivatives.
Polymeric dimethyldiallylammonium salts and copolymers thereof with acrylic acid, and esters and amides of acrylic acid and methacrylic acid. The products available commercially under the names Merquat® 100 (poly(dimethyldiallylammonium chloride)), Merquat® 550 (dimethyidiallylammonium chloride-acrylamide copolymer) and Merquat® 280 (dimethyidiallylammonium chloride-acrylic acid copolymer are examples of such cationic polymers.
Copolymers of vinylpyrrolidone with quaternized derivatives of dialkylaminoacrylate and
   methacrylate, such as, for example, vinylpyrrolidone-dimethylaminomethacrylate copolymers quaternized with diethyl sulfate. Such compounds are commercially available under the names Gafquat® 734 and Gafquat® 755.
Vinylpyrrolidone-methoimidazolinium chloride copolymers, as are supplied under the name Luviquat®.
Quaternized polyvinyl alcohol
   and the polymers known under the names
Polyquaternium 2,
Polyquaternium 17,
Polyquaternium 18 and
Polyquaternium 27 with quaternary nitrogen atoms in the polymer main chain.

Particular preference is given to cationic polymer from the first four groups, and very particular preference is given to polyquaternium-2, polyquaternium-10 and polyquaternium-22.

Also suitable as conditioning active ingredients are silicone oils, in particular dialkyl- and alkylarylsiloxanes, such as, for example, dimethylpolysiloxane and methylphenylpolysiloxane, and alkoxylated and quaternized analogues thereof. Examples of such silicones are the products sold by Dow Coming under the names DC 190, DC 200, DC 344, DC 345 and DC 1401, and the commercial products Q2-7224 (manufacturer: Dow Coming; a stabilized trimethylsilylamodimethicone), Dow Corning® 929 emulsion (comprising a hydroxylamino-modified silicone, which is also referred to as amodimethicone), SM-2059 (manufacturer: General Electric), SLM-55067 (manufacturer: Wacker), and Abil®-Quat 3270 and 3272 (manufacturer: Th. Goldschmidt; diquaternary polydimethylsiloxanes, quaternium-80).

As conditioning active ingredients it is likewise possible to use paraffin oils, synthetically prepared oligomeric alkenes, and vegetable oils, such as jojoba oil, sunflower oil, orange oil, almond oil, wheatgerm oil and peach kernel oil, and also tocopherol acetate.

Equally suitable hair-conditioning compounds are phospholipids, for example soya lecithin, egg lecithin and cephalins.

Further active ingredients, auxiliaries and additives are, for example,
nonionic polymers, such as, for example, vinylpyrrolidone/vinyl acrylate copolymers, polyvinylpyrrolidone and vinylpyrrolidone/vinyl acetate copolymers and polysiloxanes,
zwitterionic and amphoteric polymers, such as, for example, acrylamidopropyltrimethylammonium chloride/acrylate copolymers and octylacrylamide/methyl methacrylate/tert-butylaminoethyl methacrylate/2-hydroxypropyl methacrylate copolymers,
anionic polymers, such as, for example, polyacrylic acids, crosslinked polyacrylic acids, vinyl acetate/crotonic acid copolymers, vinylpyrrolidone/vinyl acrylate copolymers, vinyl acetate/butyl maleate/isobornyl acrylate copolymers, methyl vinyl ether/maleic anhydride copolymers and acrylic acid/ethyl acrylate/N-tert-butylacrylamide terpolymers,
thickeners, such as agar agar, guar gum, alginates, xanthan gum, gum Arabic, karaya gum, carob seed grain, linseed gums, dextrans, cellulose derivatives, e.g., methylcellulose, hydroxyalkylcellulose and carboxymethylcellulose, starch fractions and derivatives, such as amylose, amylopectin and dextrins, clays, such as, for example, bentonite or completely synthetic hydrocolloids, such as, for example, polyvinyl alcohol,
structurants, such as maleic acid and lactic acid,
protein hydrolysates, in particular elastin, collagen, keratin, milk protein, soya protein and wheat protein hydrolysates, condensation products thereof with fatty acids, and quaternized protein hydrolysates,
perfume oils,
cyclodextrins,
solvents and solubility promoters, such as ethanol, isopropanol, ethylene glycol, propylene glycol, glycerol, dimethyl isosorbide and diethylene glycol,
quaternized amines, such as methyl-1-alkylamidoethyl-2-alkylimidazolinium methosulfate
antifoams, such as silicones,
dyes for coloring the agent,
anti-dandruff active ingredients, such as piroctone olamine, zinc omadine and climbazole,
photoprotective agents, in particular derivatized benzophenones, cinnamic acid derivatives and triazines,
substances for adjusting the pH, such as, for example, customary acids, in particular food acids and bases,
active ingredients, such as allantoin, pyrrolidonecarboxylic acids and salts thereof, and bisabolol,
cholesterol,
consistency regulators, such as sugar esters, polyol esters or polyol alkyl ethers,
fats and waxes, such as spermaceti, beeswax, montan wax and paraffins,
fatty acid alkanolamides,
complexing agents, such as EDTA, NTA, β-alaninediacetic acid and phosphonic acids,
swelling and penetration substances, such as glycerol, propylene glycol monoethyl ether, carbonates, hydrogen carbonates, guanidines, ureas, and primary, secondary and tertiary phosphates,
opacifiers, such as latex, styrene/PVP and styrene/acrylamide copolymers,
pearlizing agents, such as ethylene glycol mono- and stearate, and PEG-3 distearate,
pigments,
stabilizers for hydrogen peroxide and other oxidizing agents,
propellants, such as propane/butane mixtures, N₂O, dimethyl ether, CO₂ and air,
antioxidants.

With regard to further optional components and the amounts of these components used, reference is expressly made to the relevant handbooks known to the person skilled in the art, e.g., Kh. Schrader, Grundlagen und Rezepturen der Kosmetika [Fundamentals and formulations of cosmetics], 2^{nd} Edition, Hüthig Buch Verlag, Heidelberg, 1989.

The present invention fourthly provides a method of lightening keratin-containing fibers, in which the fibers are treated with one of the agents described above.

The EXAMPLES below serve to illustrate the subject-matter of the present invention, but without limiting it.

### EXAMPLES

### Example 1

### Preparation of the Bleaching Agents

As component A, the following cream bases A and B as in TABLE 1 were prepared:

TABLE 1

| (according to the invention) | Cream Base A | Cream Base B |
|---|---|---|
| Hydrenol ® D¹ | 11.00% by wt. | 11.00% by wt. |
| Kokoslorol ® C12-18² | 2.20% by wt. | 2.20% by wt. |
| Texapon ® NSO F³ | 26.50% by wt. | 26.50% by wt. |
| Ammonium sulfate | 1.00% by wt. | 1.00% by wt. |
| Ammonia (25% strength aqueous | 7.60% by wt. | 7.60% by wt. |
| solution) | | |
| Gluadin ® W40⁴ | 0.35% by wt. | 0.35% by wt. |
| Sodium waterglass 40/42 | — | 2.00% by wt. |
| Perfume | 0.90% by wt. | 0.90% by wt. |
| Water | ad 100 | ad 100 |

¹C₁₆-C₁₈-fatty alcohol (INCI name: Cetearyl alcohol) (COGNIS).
²C₁₂-C₁₈-fatty alcohol (INCI name: Coconut alcohol) (COGNIS).
³Lauryl ether sulfate, sodium salt (about 27.5% active substance; (INCI name: Sodium Laureth Sulfate) (COGNIS).
⁴Wheat protein hydrolysate (INCI name: Aqua (Water), Hydrolyzed Wheat Protein, Sodium Benzoate, Phenoxyethanol, Methylparaben, Propylparaben) (COGNIS).

Prior to application to the hair, 12.5 g of ammonium peroxodisulfate were mixed into cream base A, or 12.5 g of ammonium peroxodisulfate and 5.0 g of sodium waterglass 40/42 were mixed into 50 g of cream base B. The two resulting mixtures were each admixed with 50 g of component B as in table 2 to give bleaching agent I or II, respectively. Finally, the pH was adjusted to pH 6.1 using an aqueous solution of 50% by weight citric acid.

| | Bleaching Agent II |
|---|---|
| Bleaching agent I | (according to the invention) |
| 50 g cream base A | 50 g cream base B |
| 12.5 g ammonium peroxydisulfate | 12.5 g ammonium peroxydisulfate |
| | 5.0 g sodium waterglass 40/42 |
| 50 g component B | 50 g component B |
| Citric acid solution | Citric acid solution |
| (ad pH 6.1) | (ad pH 6.1) |

TABLE 2

| | | |
|---|---|---|
| Dipicolinic acid | 0.10% | by wt. |
| Na₂H₂P₂O₇ | 0.03% | by wt. |
| Turpinal ® SL⁵ | 1.50% | by wt. |
| Texapon ® N 28⁶ | 2.00% | by wt. |
| Dow Corning DB 110⁷ | 0.07% | by wt. |
| Aculyn ® 33⁸ | 12.00% | by wt. |
| Hydrogen peroxide | 11.20% | by wt. |
| Ammonia (25% strength aqueous solution) | 0.65% | by wt. |

| | |
|---|---|
| Water | ad 100 |

⁵1-Hydroxyethane-1,1-diphosphonic acid (about 60% active substance; INCI name: Etidronic Acid) (Cognis).
⁶Sodium lauryl ether sulfate (about 28% active substance; INCI name: Sodium Laureth Sulfate) (Cognis).
⁷Nonionogenic silicone emulsion (about 10% active substance; INCI name: Dimethicone) (Dow Corning).
⁸Aqueous acrylate dispersion (about 28% active substance; INCI name: Acrylates Copolymer) (Rohm & Haas).

### Example 2

### Application and Colorimetry Measurements

The degree of lightening was determined on standardized hair swatches (Alkinco 6634) by colorimetric measurements of the fibers. The bleaching agents prepared in point 1.0 are each treated with a hair swatch for 15 minutes at 32° C. and then rinsed with water. The hair was finally dried and measured calorimetrically using the Texflash DC 3881 instrument from Datacolor to determine the CIE-Lab values.

The lightness differences of the lightened hair swatches arise from the particular CIE-Lab values as AL and the overall color change in the hair as color difference ΔE in accordance with the color difference formula given below (see also DIN 6174, German standards, Benth Verlag GmbH, Berlin, 1975). The values for the untreated starting hair serve as reference Lab values.
ΔE=[(ΔL)²+(Δa)²+(Δb)²]^{1/2} (color difference formula)

TABLE 3 summarizes the measured CIE-Lab values. The greater the ΔE value, the greater the color change compared with the starting hair. The greater the L value or the ΔL value, the higher the lightening power.

TABLE 3

| | L | a | b | ΔL | ΔE |
|---|---|---|---|---|---|
| Bleaching agent A | 45.63 | 45.63 | 24.01 | 10.68 | 15.44 |
| Bleaching agent B | 54.92 | 11.48 | 28.56 | 19.97 | 25.26 |
| (according to the invention) | | | | | |

The experiment results demonstrate that the lightening power of a bleaching agent is increased 1.87-fold by adding sodium waterglass 40/42 as optionally hydrated SiO₂ compound.

1. A method for increasing the power of bleaching agents to lighten the color of keratin fibers, said method comprising the step of treating the fibers with bleaching agents comprising at least one optionally hydrated SiO₂ compound.

2. The method as claimed in claim 1, characterized in that the optionally hydrated SiO₂ compound is a silica gel.

3. The method as claimed in claim 1, characterized in that the optionally hydrated SiO₂ compound is a silicate of the formula (SiO₂)ₙ(Na₂O)ₘ(K₂O)ₚ, where n is a positive whole number and m and p, independently of one another, are a positive whole number or are 0, with the provisos that at least one of the parameters n or p is different from 0 and the ratio between n and the sum of m and p is between 1:4 and 4:1.

4. The method as claimed in claim 3, characterized in that the optionally hydrated SiO₂ compound is an aqueous solution of the silicate of the formula (SiO₂)ₙ(Na₂O)ₘ(K₂O)ₚ, in which n, m and p are as defined in claim 3.

5. An agent for lightening keratin-containing fibers comprising at least one peroxo compound and hydrogen peroxide, characterized in that it additionally comprises at least one optionally hydrated SiO₂ compound.

6. The agent as claimed in claim 5, characterized in that it has a pH of from 4.5 to 9.0.

7. An agent for lightening human hair, comprising hydrogen peroxide, and at least one alkalinizing agent selected from the group consisting of ammonium, alkali metal and alkaline earth metal carbonates, hydrogen carbonates and carbamides, further characterized in that it comprises at least one optionally hydrated SiO₂ compound and has a pH of from 7.5 to 9.0.

8. The agent as claimed in claim 7, characterized in that it additionally comprises at least one peroxo compound.

9. The agent as claimed in claim 5, characterized in that the optionally hydrated SiO₂ compound is a silicate of the formula (SiO₂)ₙ(Na₂O)ₘ(K₂O)ₚ, where n is a positive whole number and m and p, independently of one another, are a positive whole number or are 0, with the provisos that at least one of the parameters m or p is different from 0 and the ratio between n and the sum of m and p is between 1:4 and 4:1.

10. The agent as claimed in claim 9, characterized in that the optionally hydrated SiO₂ compound is an aqueous solution of the silicate of the formula (SiO₂)ₙ(Na₂O)ₘ(K₂O)ₚ, in which n, m and p are as defined in claim 8.

11. The agent as claimed in claim 5, characterized in that the optionally hydrated SiO₂ compound is a silica gel.

12. The agent as claimed in claim 5, characterized in that it comprises the optionally hydrated SiO₂ compounds in an amount of from 0.05 to 15% by weight.

13. The agent as claimed in claim 5, characterized in that it additionally comprises at least one alkalinizing agent.

14. The agent as claimed in claim 5, characterized in that a structure-improving active ingredient selected from the group consisting of panthenol, physiologically compatible panthenol derivatives, mono-, di- and oligosaccharides, serine, glyceric acid, niacinamide, vitamin B6, polyvinylpyrrolidone, gluconic acid, biotin and the lipid-soluble ester alcohols or ester polyols is additionally present.

15. The agent as claimed in claim 14, characterized in that it comprises the structure-improving active ingredient in amounts of from 0.1 to 5% by weight.

16. The agent as claimed in claim 5, characterized in that it further comprises a magnesium compound.

17. The agent as claimed in claim 5, characterized in that the peroxo compound is selected from the group consisting of ammonium peroxydisulfate, potassium peroxydisulfate, sodium peroxydisulfate, ammonium persulfate, potassium persulfate, sodium persulfate, potassium peroxydiphosphate, magnesium peroxide and barium peroxide.

18. The agent as claimed in claim 5, characterized in that at least one acid selected from the group consisting of acetic acid, lactic acid, tartaric acid, citric acid, salicylic acid and ortho-phthalic acid is additionally present.

19. A method of lightening the color of human hair fibers comprising the step of treating the hair fibers with an agent as claimed in claim 5.

20. The method as claimed in claim 19, characterized in that the agent as claimed in claim 5, immediately prior to application to the fibers, is mixed with a composition A comprising at least one peroxo compound and/or a composition B comprising hydrogen peroxide, with the provisos that at least one of the compositions A and B comprises an optionally hydrated SiO₂ compound and the pH of the resulting mixture is 4.5 to 9.0

21. The method as claimed in claim 20, characterized in that at least one of the compositions A and B comprises an alkalinizing agent selected from the group consisting of ammonium, alkali metal and alkaline earth metal carbonates, hydrogen carbonates and carbamides.

22. The method as claimed in claim 20, characterized in that the pH of the resulting mixture is 7.5 to 9.0.

23. The method as claimed in claim 20, characterized in that at least one of the compositions A and B comprises at least one structure-improving active ingredient selected from the group consisting of panthenol, physiologically compatible panthenol derivatives, mono-, di- and oligosaccharides, serine, glyceric acid, niacinamide, vitamin B6, polyvinylpyrrolidone, gluconic acid, biotin and the lipid-soluble ester alcohols and ester polyols.
